(19) United States
(12) Patent Application Publication
(10) Pub. No.: US 2004/0081687 A1
(43) Pub. Date: Apr. 29, 2004

(54) Compound

(76) Inventors: Andrew David Miller, London (GB); Michael Rael Jorgensen, London (GB); Michael Keller, London (GB); Eric Perouzel, London (GB)

Correspondence Address: Scott A McCollister, Fay Sharpe Fagan Minnich & McKee, 7th Floor, 1100 Superior Avenue, Cleveland, OH, 44114-2518, US

(21) Appl. No.: 10/450,356

(22) PCT Filed: Dec. 5, 2001
(86) PCT No.: PCT/GB01/05383

(30) Foreign Application Priority Data
Dec. 12, 2000 (WO) PCT/GB00/04767
Jun. 6, 2001 (GB) 0113781.9

(51) Int. Cl. C07J 1/00; A61K 48/00; A61K 9/127

(52) U.S. Cl. 424/450; 514/44; 536/5

(57) ABSTRACT

The present invention provides a compound capable of acting as a cationic lipid, the compound comprises a cholesterol group and a carbohydrate moiety.

The present invention relates to a compound. In addition, the present invention relates to processes for making the compound and to the use of that compound in therapy, in particular gene therapy (especially gene transfer).

One aspect of gene therapy involves the introduction of foreign nucleic acid (such as DNA) into cells, so that its expressed protein may carry out a desired therapeutic function.^{1a}

Examples of this type of therapy include the insertion of TK, TSG or ILG genes to treat cancer, the insertion of the CFTR gene to treat cystic fibrosis; the insertion of NGF, TH or LDL genes to treat neurodegenerative and cardiovascular disorders; the insertion of the IL-1 antagonist gene to treat rheumatoid arthritis; the insertion of HIV antigens and the TK gene to treat AIDS and CMV infections; the insertion of antigens and cytokines to act as vaccines; and the insertion of β-globin to treat haemoglobinopathic conditions, such as thalassaemias.

Many current gene therapy studies utilise adenoviral gene vectors—such as Ad3 or Ad5—or other gene vectors. However, serious problems have been associated with their use.^{2a} This has prompted the development of less hazardous, nonviral approaches to gene transfer.^{3a}

A nonviral transfer system of great potential involves the use of cationic liposomes.^{4a} In this regard, cationic liposomes—which usually consist of a neutral phospholipid and a cationic lipid—have been used to transfer DNA^{4a}, mRNA^{5a}, antisense oligonucleotides^{6a}, proteins^{7a}, and drugs^{8a} into cells. A number of cationic liposomes are commercially available^{4a,9a} and many new cationic lipids have recently been synthesised^{10a}. The efficacy of these liposomes has been Illustrated by both in vitro^{4a} and in vivo^{11a}.

A neutral phospholipid useful in the preparation of a cationic liposome is N-[1-(2,3-dioleoyloxy)propyl]-N,N,N-trimethyl ammonium chloride, otherwise known as “DOTMA”.

One of the most commonly used cationic liposome systems consists of a mixture of a neutral phospholipid diol oylphosphatidylethanolamine (commonly known as “DOPE”) and a cationic lipid, 3β-[(N,N-dimethylaminoethyl)carbamoyl]cholesterol (commonly known as “DC-Chol”)^{12a}.

Despite the efficacy of the known cationic liposomes there is still a need to optimise the gene transfer efficiency of cationic liposomes in human gene therapy^{10a}. With the near completion of the human genome project, the use of genes for therapeutic purposes, described as gene therapy Is increasingly expected to revolutionise medicine. In this context, even though still less effective than viral technology, non-viral delivery is increasingly recognised by the scientific community as the safest option for human applications.

This field has evolved considerably in the last decade with the apparition of complex macromolecular constructs including many elements of different existing technologies (viral proteins or peptides, liposomes, polymers, targeting strategies and stealth properties).

Our copending application PCT/GB00/04767 teaches a system based on a triplex composed of a viral core peptide Mu, plasmid DNA and cationic Liposome (LMD). This platform technology gave us good success in vitro and promising results in vivo. But as for all existing non-viral technology more development is needed to achieve a therapeutic level in vivo.

To this end, formulation must achieve stability of the particle in biological fluids (serum, lung mucus) and still maintain efficient transfection abilities.

This requirement is one of the main hurdles of all existing technology. Current stable formulations ^{[1, 2]} achieve little transfection and most present efficient transfecting agents are drastically limited in the scope of their application due to this instability ^{[3-7]}.

After administration (in blood for systemic application or in mucus for lung topical administration), the charged complexes are exposed to salt and biological macromolecules leading to strong colloidal aggregation and adsorption of biological active elements (opsonins) at their surface^{[8-11]}. The gene vehicles undergo drastic changes that could include precipitation, binding of proteins leading to particle elimination by macrophages and surface perturbation resulting in its destruction. The most widely used stabilised formulation involves surface-grafted polyethylene glycol (PEG) chains^{[12,13]} PEG is a non-toxic, neutral polyether which has a large exclusion volume for most macromolecules. Unfortunately formulations demonstrating the necessary level of stabilisation loose their gene transfer ability; probably due to their reduced non-specific affinity for cells or the loss of their necessary endosome breaking properties^{[14, 15]}.

An alternative approach to escaping the destructive effect of biological fluid on lipoplexes is to attempt to mimic nature and coat the surface of lipid bilayers with polysaccharides^{[16,17]}.

The present invention alleviates the problems of the prior art.

According to one aspect of the present Invention there is provided a compound capable of acting as a cationic lipid, the compound comprises a cholesterol group and a carbohydrate moiety.

According to another aspect of the present invention there is provided a process of preparing a compound according to the present invention comprising reacting a compound comprising a cholesterol group and a polyamine with a saccharide.

According to another aspect of the present invention there is provided a compound according to the present invention or a compound when prepared by the process of the present invention for use in therapy.

According to another aspect of the present invention there is provided the use of a compound according to the present invention or a compound when prepared by the process of the present invention in the manufacture of a medicament for the treatment of a genetic disorder or a condition or a disease.

According to another aspect of the present invention there is provided a cationic liposome formed from the compound according to the present invention or a compound when, prepared by the process of the present invention.

According to another aspect of the present invention there is provided a method of preparing a cationic liposome comprising forming the cationic liposome from the compound according to the present invention or a compound when prepared by the process of the present invention.

According to another aspect of the present invention there is provided a cationic liposome according to the present invention or a cationic liposome as prepared by the method of the present invention for use in therapy.

According to another aspect of the present invention there is provided the use of a cationic liposome according to the present invention or a cationic liposome as prepared by the method of the present invention in the manufacture of a medicament for the treatment of genetic disorder or condition or disease.

According to another aspect of the present invention there is provided a combination of a nucleotide sequence and any one or more of: a compound according to the present invention, a compound when prepared by the process of the present invention, a liposome of the present invention, or a liposome as prepared by the method of the present invention.

According to another' aspect of the present invention there is provided a combination according to the present invention for use in therapy.

According to another aspect of the present invention there is provided the use of a combination according to the present invention in the manufacture of a medicament for the treatment of genetic disorder or condition or disease.

According to another aspect of the present invention there Is provided a pharmaceutical composition comprising a compound according to the present invention or a compound when prepared by the process of the present invention admixed with a pharmaceutical and, optionally, admixed with a pharmaceutically acceptable diluent, carrier or excipient.

According to another aspect of the present Invention there is provided a pharmaceutical composition comprising a cationic liposome according to the present invention or a compound liposome as prepared by the method of the present invention admixed with a pharmaceutical and, optionally, admixed with a pharmaceutically acceptable diluent, carrier or excipient.

Some further aspects of the invention are defined in the appended claims.

It is believed that a key advantage of the compound of the present invention is that it can be used as a cationic lipid (amphiphile) in the preparation of a cationic liposome useful in gene therapy, in particular the transfer of nucleic acids (including genes and antisense DNA/RNA) into cells (in vitro, in vivo and ex vivo) to derive a therapeutic benefit.

Carbohydrates have numerous biological functions. We have exploited their combined targeting potential and stabilisation properties^{[18-20]}. We have designed a glycolipid family based on a previously developed cholesterol based cationic lipid to Insert properly into the bilayer. To evaluate the minimum size of the carbohydrate motif needed to stabilise our system, a chemoselective methodology^{[21-23]} was chosen allowing a facile modulation of the number of glycosidic units^{[24-26]}. The key step exploited the formation of an oxime bond for the attachment of lipids to aldehyde-containing compounds such as simple carbohydrates. In sharp contrast to other methods applied for synthesis of glycolipids, this procedure permits preservation of the cyclic nature of the saccharide unit with high efficiency, is more simple than traditional methods and does not require extensive protection group manipulation for each new sugar coupled as long as an aldehyde form exists (mutarotation equlilbrium).

Thus according to another aspect of the present invention there is provided a process of preparing a compound according to the present invention comprising reacting a compound comprising a cholesterol group and a polyamine group with an unprotected saccharide.

Preferred Aspects

In a preferred aspect the compound of the invention is of the formula Chol-Carb wherein Chol is a cholesterol group, L is an optional linker group and Carb is a carbohydrate moiety.

In a preferred aspect the cholesterol group Is cholesterol.

In a preferred aspect the cholesterol group is linked to the optional linker group via a carbamoyl linkage.

In a highly preferred aspect the compound of the present invention is of the formula Chol-L-Carb, wherein Chol Is cholesterol, L Is a polyamine group and Carb is a glucose, preferably D-glucose.

Linker

In a preferred aspect the linker group is a polyamine group. It Is believed that the polyamine group is advantageous because it Increases the DNA binding ability and efficiency of gene transfer of the resultant liposome.

In one embodiment, preferably the polyamine group is a naturally occurring polyamine. It is believed that the polyamine headgroup is advantageous because the increased amino functionality increases the overall positive charge of the liposome. In addition, polyamines are known to both strongly bind and stabilise DNA^{14a}. In addition, polyamines occur naturally in cells and so it is believed that toxicological problems are minimised^{15a}.

In another embodiment, preferably two or more of the amine groups of the polyamine group of the present invention are separated by one or more groups which are not found in nature that separate amine groups of naturally occurring polyamine compounds (i.e. preferably the polyamine group of the present invention has un-natural spacing).

Preferably the polyamine group contains at least two amines of the polyamine group that are separated (spaced from each other) from each other by an ethylene (—CH₂CH₂—) group.

Preferably each of the amines of the polyamine group are separated (spaced from each other) by an ethylene (—CH₂CH₂—) group.

Typical examples of suitable polyamines include spermidine, spermine, caldopentamine, norspermidine and norspermine. Preferably the polyamine is spermidine or spermine as these polyamines are known to interact with single or double stranded DNA. An alternative preferred polyamine is caldopentamine.

In a preferred aspect the linker group is a polyethylene glycol (PEG) group. The PEG group preferably contains from 4 to 16 oxyethylene units In size, for example 4, 6, 8, 10, 12, 14 or 16 oxyethylene units.

In a preferred aspect the linker group contains (PEG) group and a polyamine group. In a preferred aspect the linker group is a conjugate of oxyethylene groups and amine groups. In either of these aspects the preferred features of the PEG groups and polyamine groups disclosed above equally apply.

Carbohydrate

In a preferred aspect the carbohydrate moiety is a mono-saccharide.

In a preferred aspect the carbohydrate moiety is a sugar moiety.

Preferably the carbohydrate moiety is selected from mannose, glucose (D-glucose), galactose, glucuronic acid, lactose, maltose, maltotriose, maltotetraose, maltoheptaose and mixtures thereof. More preferably the carbohydrate moiety Is D-glucose.

In one aspect the compound of the present invention comprises from 1 to 7 carbohydrate moieties. Preferably the compound comprises one carbohydrate moiety.

Cholesterol

The cholesterol group can be cholesterol or a derivative thereof. Examples of cholesterol derivatives include substituted derivatives wherein one or more of the cyclic CH₂ or CH groups and/or one or more of the straight-chain CH₂ or CH groups is/are appropriately substituted. Alternatively, or in addition, one or more of the cyclic groups and/or one or more of the straight-chain groups may be unsaturated.

In a preferred embodiment the cholesterol group is cholesterol. It is believed that cholesterol is advantageous as it stabilises the resultant liposomal bilayer.

Preferably the cholesterol group is linked to the optional linker group via a carbamoyl linkage. It Is believed that this linkage is advantageous as the resultant liposome has a low or minimal cytotoxicity.

Further Aspects

Preferably the compound is in admixture with or associated with a nucleotide sequence.

The nucleotide sequence may be part or all of an expression system that may be useful In therapy, such as gene therapy.

In a preferred aspect the compound of the present invention Is in admixture with a condensed polypeptide/nucleic acid complex to provide a non-viral nucleic acid delivery vector. The condensed polypeptide/nucleic acid complex preferably include those disclosed in our copending application PCT/GB00/04767. Preferably the polypeptides or derivatives thereof are capable of binding to the nucleic acid complex. Preferably the polypeptides or derivatives thereof are capable of condensing the nucleic acid complex. Preferably the nucleic acid complex is heterologous to the polypeptides or derivatives thereof.

Preferably the process comprises the use of a molecular sieve.

Preferably, the cationic liposome is formed from the compound of the present invention and a neutral phospholipid—such as DOTMA or DOPE. Preferably, the neutral phospholipid is DOPE.

In one aspect the saccharide is attached to the polyamine group via a terminal amine of the polyamine. In other words a primary amine of the polyamine is substituted.

In summation, the present invention provides a compound capable of acting as a cationic lipid, the compound comprises a cholesterol group and a carbohydrate moiety.

A preferred embodiment of the present invention is a compound capable of acting as a cationic lipid, the compound comprising a cholesterol group having linked thereto via a polyamine group, a saccharide.

A more preferred embodiment of the present invention is a compound capable of acting as a cationic lipid, the compound comprising a cholesterol group having glucose linked thereto via a polyamine group.

A highly preferred embodiment of the present invention is a compound capable of acting as a cationic lipid, the compound comprising cholesterol having glucose (preferably D-glucose) linked thereto via a polyamine group.

In one aspect of the present invention the saccharide of the present Invention may be fully or partially substituted by a polyethylene glycol (PEG). Thus In further aspects the present invention provides

a compound capable of acting as a cationic lipid, the compound comprises a cholesterol group and a polyethylene glycol moiety.

a compound capable of acting as a cationic lipid, the compound, comprising a cholesterol group having linked thereto via a polyamine group, a polyethylene glycol.

a compound capable of acting as a cationic lipid, the compound comprising a cholesterol group having polyethylene glycol linked thereto via a polyamine group.

a compound capable of acting as a cationic lipid, the compound comprising cholesterol having polyethylene glycol linked thereto via a polyamine group.

In one aspect the cationic lipid of the present invention is modified with a sugar moiety or a polyethylene glycol (PEG) moiety. In a further aspect the complex of the invention further comprises a compound capable of acting as a cationic lipid, the compound comprising a cholesterol group having linked thereto via an amine group, a sugar moiety or a polyethylene glycol moiety. As demonstrated in the Examples we have found such sugar/PEG modified cationic lipids to be particularly advantageous. Thus in a further aspect the present invention provides a compound capable of acting as a cationic lipid, the compound comprising a cholesterol group having linked thereto via an amine group, a sugar moiety or a polyethylene glycol moiety. Preferably the compound comprises from 1 to 7 sugar moieties or a polyethylene glycol moieties. The compound may comprise a mixture of sugar moieties and polyethylene glycol moieties. Preferably the sugar moiety is or is derived from glucose or D-glucose.

The present invention will now be described in further detail by way of example only with reference to the accompanying figures n which:—

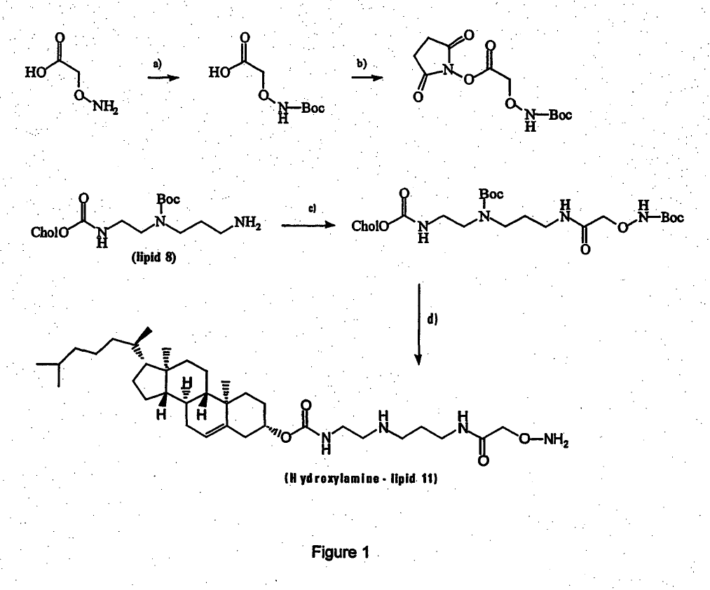

FIG. 1—Scheme 1 Synthesis of Hydroxylamine lipid 11. Reagents. (a) CHC₂Cl₂, Et₃N, Boc₂O, rt, 5 h, 98%; (b) EtOAc, N-hydroxysuccinimide (1 eq.), DCC (1 eq.), 10 h., rt; (c) (8), EtOAC/THF [95/5], Et₃N (pH=8), 2 h., r.t, 90%; (d) CH₂Cl₂, TFA (15 eq), 0° C., N₂, 5 h, 86%.

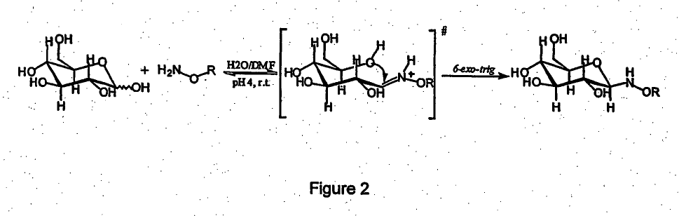

FIG. 2—Principle of chemioselective glycosylation of O-substituted hydroxylamine with D-Glucose (Although the β-anomer is shown, mutarotation does occur and α-anomer is produced as well).

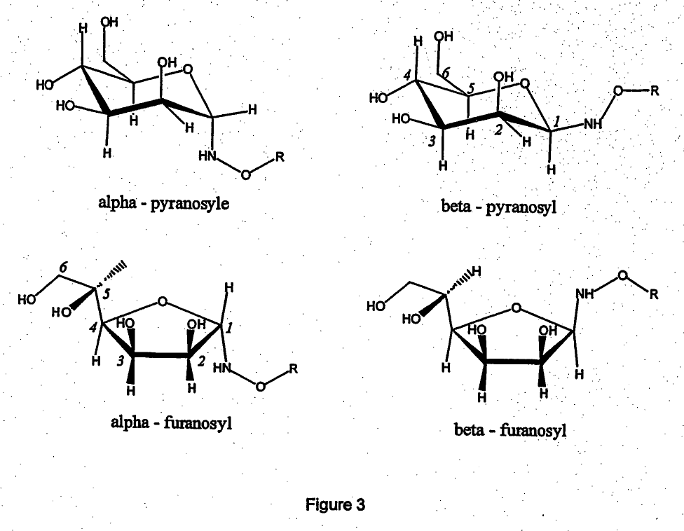

FIG. 3—Possible structures of neoglycolipid obtained from mannose.

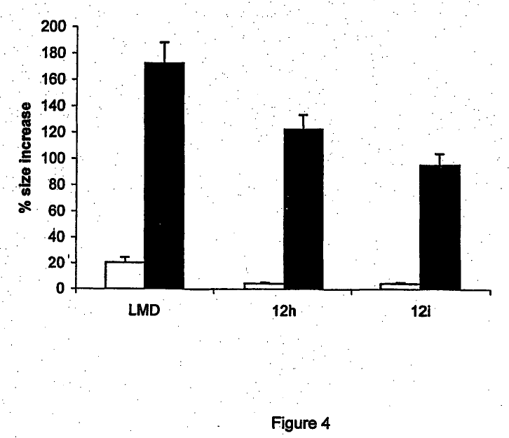

FIG. 4—Result of analysis of differents lipoplexes size by photon correlation spectroscopy (PCS). The size was measured after 30 min for lipoplexes at [DNA]=1 μg/ml In Optimem ±10% FCS, 37° C. The comparison of standard LMD formulation (LMD) and LMD modified by addition of 7.5 molar % of product 12h and 12l was made In Optimem (white) and 10% Serum (black) and expressed In percent of size. increase over the original measured size of 180 nm.

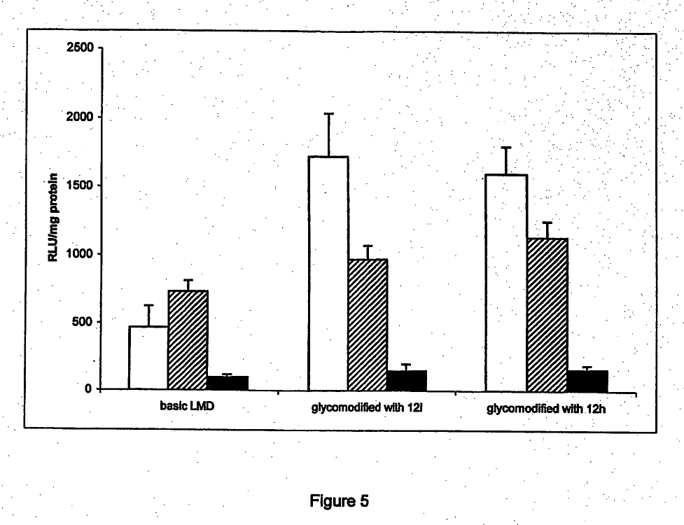

FIG. 5—A comparison between the transfection efficiencies of basic LMD and LMD glycomodified with 7.5 molar % of product 12h and 12l onto Hela Cells in 0% (white), 50% (black and white) and 100% Serum (black) conditions. The results are expressed as relative light units per milligram of protein (n=4).

The present invention will now be described in further detail in the following examples.

### EXAMPLES

### Experimental Section

### Synthesis of Neoglycolipids

General: ¹H NMR spectra were recorded at ambient temperature on either Brucker DRX400, DRX300 or Jeol GX-270Q spectrometers, with residual nonisotopicaly labeled solvent (e.g. CHCl3, δ_{H}=7.26) as an internal reference. ¹³C-NMR spectra were recorded on the same range of spectrometers at 100, 75 and 68.5 MHz respectively, also with residual nonisotopicaly labelled solvent (e.g. CHCl₃, δ_{d}=77.2) as an internal reference. Infrared Spectra were recorded on Jasco FT/IR 620 using NaCl plates and Mass spectra (Positive ions electrospray) were recorded using VG-7070B or JEOL SX-102 instruments. Chromatography refers to flash column chromatography, which was performed throughout on Merck-Kieseigel 60 (230-400 mesh) with convenient solvent.

Thin layer chromatography (Tlc) was performed on pre-coated Merck-Kieseigel 60 F254 aluminium backed plated and revealed with ultraviolet light, iodine, acidic ammonium molybdate(IV), acidic ethanolic vanilin, or other agents as appropriate. Neoglycolipids purity was assessed using analytical, high-pressure liquid chromatography (HPLC) on a Hitachi system using a Purospher® RP-18 endcapped column (5 μm). Elution was performed at an isocratic flow rate of 1 mL/min with CH₃CN/H₂O (60:40) and fraction were detected at 205 nm wavelength before collection and Mass Analysis. Dried CH2Cl2 was distilled with phosphorous pentoxide before use. All other dry solvents and chemicals were purchased from Sigma-Aldrich Company LTD (Poole, Dorset, UK).

Abbreviations: Boc: tert-butoxycarbonyl; br: broad; Chol: cholesteryl; DMF: N,N-dimethyl formamide; DMSO: dimethyl sulfoxide; TFA: trifluoroacetic acid; THF: tetrahydrofuran.

2-(Cholesteryloxycarbonyl)aminoethanol (2): A solution of cholesteryl chloroformate (99.89 g, 0.218 mol) in CH₂Cl₂ (600 mL) was added to a stirred solution of 2-aminoethanol (29.5 mL, 0.489 mol, 2.2 equiv) in CH₂Cl₂ (450 mL) at 0° C. over a period of 2 hours. The reaction was allowed to warm to room temperature and stirring continued for a further 14 h. The reaction mixture was washed with saturated NaHCO₃ (2*200 mL), water (2*200 mL), dried (MgSO₄) and the solvents removed under reduced. The solid obtained was recrystallised (CH₂Cl₂/MeOH) to give 2 as a white solid. Yield: 99.67 g (97% ); m.p.: 180° C.; R_{f}=0.26 (acetone/ether 1:9); IR (CH₂Cl₂): νₘₐₓ =3353, 2942, 2870, 1693, 1674, 1562, 1467, 1382, 1264 cm⁻¹; ¹H NMR (270 MHz, CDCl₃): δ=5.35 (d, J=6.5 Hz, 1H, H6′), 5.25-5.29 (m, 1H, NH), 4.42-4.57 (1H, m, H3′), 3.70-3.62 (m, 2H, H1), 325-3.35 (m, 2H, H2), 3.12 (s, 1H, OH), 2.28-2.38 (m, 2H, H4′), 1.77-2.03 (m, 5H, H2′, H7′, H8′), 1.59-0.96 (m, 21H, H1′, H9′, H11′, H12′, H14′-H17′, H22′-H25′), 1 (3H, s, H-19′), 0.9(d, J=6.5 Hz, 3H, H21′), 0.87 (d, J=6.5 Hz, 6H, H26′&H27′) and 0.67 (s, 3H, H18′); MS (FAB⁺): m/z=496 [M+Na]⁺, 474.[M+H]⁺, 369[Chol]⁺, 255,175,145,105,95,81,43.

2-[(Cholesteryloxycarbonyl)amino]ethyl methanesulfonate (3): To a solution of 2 (25 g, 52.3 mmol) and triethylamine (22 mL, 0.16 mol, 3 equiv) in CH₂Cl₂ (500 mL) at 0° C. was added dropwise a solution of methanesulfonyl chloride (10.5 mL, 0.13 mol, 2.5 equiv). The reaction mixture was allowed to warm at room temperature and stirred for 1 h30. After Tlc analysis has Indicated that the reaction had gone to completion, ice was added to quench the reaction. The reaction mixture was added to saturated aqueous NH₄Cl (600 mL), and extracted with ether (3*300 mL). The combined organic layers were washed with water (2*300mL), brine (250 mL) and dried (Na₂SO₄). The solvent was remove under reduced pressure to give a white solid, which on purification by chromatography (ether) gave 3. Yield: 28.3 g (98%); IR (CH₂Cl₂): νₘₐₓ=3453, 3342, 1716, 1531, 1377, 1137 & 798 cm⁻¹; ¹H NMR (270 MHz, CDCl₃): δ=5.34 (d, J=6.5 Hz, 1H, H6′), 5-5.1 (m, 1H, NH), 4.41-4.53 (1H, m, H3′), 4.29-4.25 (t, J=5 Hz, 2H, H1), 3.47-3.52 (m, 2H, H2), 3.01 (s, 3H, H3), 2.24-2.36 (m, 2H, H4′), 1.74-2 (m, 5H, H2′, H7′, H8′), 0.9-1.6 (m, 21H, H1′, H9′, H11′, H12′, H14′-H17′, H22′-H25′), 0.98 (3H, s, H-19′), 0.84(d, J=6.5 Hz, 3H, H21′), 0.83 (d, J=6.5 Hz, 6H, H26′&H27′) and 0.65 (s, 3H, H18′); MS (FAB⁺): m/z=1104[2M+H]⁺, 574 [M+Na]⁺, 552 [M+H]⁺, 369[Chol]⁺, 255,175,145,95,81.

4-aza-N⁶(cholesteryloxycarbonylamino) hexanol (4): To a stirred solution of 3 (28.3 g, 51 mmol) dissolved In a minimum amount of THF, was added amino-propanol (160 mL, 2 mol, 39 equiv). Once Tlc indicated reaction completion (12 h), CHCl₃ (350 mL) and K₂CO₃ (20 g) were added and the solution was vigorously stirred for 30 min. The suspension was then filtered through a short pad of Celite®, washing thoroughly with CHCl₃. This was washed with a saturated solution of Sodium Hydrogenocarbonate and dried (Na₂CO₃). The solvent was removed to give 4 as a white solid. Yield: 26.1 g (96%); IR (CH₂Cl₂): νₘₐₓ=3350-3210, 2937, 2850, 1531, 1460, 1380, 1220, 1120, 1040 cm⁻¹; ¹H NMR (270 MHz, CDCl₃): δ=5.33-5.35 (m, 1H, H6′), 4.92-4.96 (m, 1H, NH), 4.42-4.51 (1H, m, H3′), 3.7-3.83. (m, 2H, H5), 3.23-3.29 (m, 2H, H1), 2.73-2.57 (m, 6H, H2, H3, H4), 2.2-2.36 (m, 2H, H4′), 1.7-2 (m, 5H, H2′, H7′, H8′), 0.85-1.58 (m, 21H, H1′, H9′, H11′, H12′, H14′-H17′, H22′-H25′), 0.98 (3H, s, H-19′), 0.84 (d, J=6.5 Hz, 3H, H21′), 0.8 (d, J=6.5 Hz, 6H, H26′&H27′) and 0.61 (s, 3H, H18′); MS (FAB⁺): m/z=543 [M+Na]⁺, 530 [M+H]⁺, 485 [M−CO₂]⁺, 369[Chol]⁺, 144 [M−Ochol]⁺, 69,55.

4-aza-(Boc)-N⁶(cholosteryloxycarbonyl amino) hexanol (5): To a solution of 4 (26.1 g, 49 mmol), was added Et3N (8.3 mL, 1.1 equiv) and Boc₂O (10.7 g, 1 equiv) in CH₂Cl₂ (200 mL) and the resulting solution followed by tlc. On completion, the reaction mixture was poured into NH₄Cl (100 mL), and was washed with water and dried (Na₂SO₄). The solvent was removed in vacuo to give the white solid 5. The solvent was remove under reduced pressure to give a white solid, which on purification by chromatography (CH₂Cl₂/M OH/NH₃ 92:7:1) gave 3. Yield (27.9 g, 90% ); IR (CH₂Cl₂): νₘₐₓ=3352, 3054, 2937, 1675, 1530,1455, 1380, 1220, 1120; ¹H NMR (270 MHz, CDCl₃): δ=5.33-5.35 (m, 1H, H6′), 4.86 (m, 1H, NH), 4.42-4.5 (1H, m, H3′), 3.62-3.7 (m, 2H, H5), 3.27-3.38 m, 6H, H1, H2, H3), 2.18-2.33 (m, 2H, H4′), 1.73-2 (m, 5H, H2′, H7′, H8′), 1.45 (s, 9H, Boc), 1-1.65 (m, 23H, H4, H1′, H9′, H11′, H12′, H14′-H17′, H22′-H25′), 0.97 (3H, s, H-19′), 0.93 (d, J=6.5 Hz, 3H, H21′), 0.8 (d, J=6.5 Hz, 6H, H26′&H27′) and 0.65 (s, 3H, H18′); MS (FAB⁺): m/z=654 [M+Na]⁺, 543 [M−Boc]⁺, 369[Chol]⁺, 145, 121, 95, 69,57.

4-aza-(Boc)-N⁶(cholesteryloxycarbonylamino) hexyl methane-sulfonate (6): This experiment was carried out in a similar way as the preparation of 2-[(Cholesteryloxycarbonyl)amino]ethyl methanesulfonate 3 on 44 mmol scale giving 6. Yield (28 g, 90% ); IR (CH₂Cl₂): νₘₐₓ=3305, 2980, 2900, 2865, 1675, 1530, 1455, 1350, 1150; ¹H NMR (270 MHz, CDCl₃): δ=5.33-5.35 (m, 1H, H6′), 4.86 (m, 1H, NH), 4.354.55 (m, 1H, H3′), 4.22 (t, 2H, J=6.5 Hz, H5), 3.2-3.4 (m, 6H, H1, H2, H3), 3.01(s, 3H, H6), 2.15-2.33 (m, 2H, H4′), 1.73-2 (m, 5H, H2′, H7′, H8′), 1.44 (s, 9H, Boc), 1-1.67 (m, 23H, H4, H1′, H9′, H11′, H12′, H14′-H17′, H22′-H25′), 0.97 (3H, s, H-19′), 0.94 (d, J=6.5 Hz, 3H, H21′), 0.8 (d, J=6.5 Hz, 6H, H26′&H27′) and 0.65 (s, 3H, H18′); MS-(FAB⁺): m/z=722 [M+Na]⁺, 609 [M−Boc]⁺, 369[Chol]⁺, 145,121, 95, 69,55.

4-aza-(Boc)-N⁶(cholesteryloxycarbonylamino) hexanamine (7): To 6 (25 g, 35 mmol), sodium azide (11.49, 175.7 mmol, 5 equiv), and sodium iodine (5 g, 35 mmol, 1 equiv) under nitrogen was added anhydrous DMF (200 mL), with stirring. Equipped with a reflux condenser, heating at 80° C. for 2 h resuited in completion of reaction. The reaction mixture was allowed to cool to room temperature, the DMF removed under reduced pressure and the residue dissolved in EtOAc. This was washed with water (2*100 mL), brine (100 mL) and dried (Na₂SO₄) to give after purification by chromatography (hexane/ether 1:1) 7 as a white solid. Yield.(22 g, 95% ); ¹H NMR (270 MHz, CDCl₃): δ=5.34-5.36 (m, 1H, H6′), 4.35-4.55 (m, 1H, H3′), 4.25 (t, 2H, J=6.5 Hz, H5), 3.2-3.5 (m, 6H, H1, H2, H3), 2.25-2.33 (m, 2H, H4′), 1.7-2.05 (m, 5H, H2′, H7′, H8′), 1.45 (s, 9H, Boc), 1-1.72 (m, 23H, H4, H1′, H9′, H11′, H12′, H14′-H17′, H22′-H25′), 0.98 (3H, s, H-19′), 0.94 (d, J=6.5 Hz, 3H, H21′), 0.83 (d, J=6.5 Hz, 6H, H26′&H27′) and 0.64 (s, 3H, H18′); MS (FAB⁺): m/z=568 [M+Na−Boc]⁺, 556 [M−Boc]⁺, 369[Chol]⁺, 145, 121, 95, 69,57.

4-aza-(Boc)-N⁶(cholesteryloxycarbonylamino) hexylamine (8): To a round bottomed flask charged with 7 (22.75 g, 34,6 mmol) in THF (230 mL) was added trimethylphosphine in THF (1 M, 40 mL, 1.15 equiv), and the reaction was monitored by tlc. On the completion the reaction was stirred with water (3 mL) and aqueous ammonia (3 mL) for 1 h and the solvent was remove under reduce pressure. After chromatography (CH₂Cl₂/MeOH/NH₃ 92:7:1 to 75:22:3) 8 was obtained as a white crystal. Yield (19.1 g, 88% ); IR (CH₂Cl₂): νₘₐₓ=3689, 3456, 3155, 2948, 2907, 2869, 2253, 1793, 1709, 1512, 1468, 1381, 1168; ¹H NMR (270 MHz, CDCl₃):δ=5.32-5.35 (m, 1H, H6′), 4.35-4.51 (m, 1H, H3′), 3.4-3.05 (m, 8H, H1, H2, H3, H5), 2.18-2.4 (m, 2H, H4′), 1.82-2.1 (m, 5H, H2′, H7′, H8′), 1.46 (s, 9H, Boc), 1.01-1.72 (m, 23H, H4, H1′, H9′, H11′, H12′, H14′-H17′, H22′-H25′), 0.97 (3H, s, H-19′), 0.85 (d, J=6.5 Hz, 3H, H21′), 0.82 (d, J=6.5 Hz, 6H, H26′&H27′) and 0.64 (s, 3H, H18′); MS (FAB⁺): m/z=630 [M+H]⁺, 530 [M−Boc]⁺, 369[Chol]³⁰ , 145, 121, 95, 69,57.

(Boc)aminooxyacetic acid (9): O-(Carboxymethyl)hydroxylamine hemihydrochloride (1.16 g, 5.3 mmol) was dissolved in CH₂Cl₂ (40 mL) and the pH was adjusted to 9 by addition of triethylamine (3 mL). Then di-tert-butyl dicarbonate (2.36 g, 10.6 mmol, 2.0 equiv) was added and the mixture was stirred at room temperature until tlc indicated completion of reaction. The pH was lowered to 3 by addition of diluted HCl. The reaction mixture was partitioned between saturated aqueous NH₄Cl (20 mL) and CH₂Cl₂ (30 mL). The aqueous phase was extracted with CH₂Cl₂ (3×100 mL). The combined organic extracts were washed with H₂O (2×100 mL) and dried (Na2SO₄). The solvent was removed in vacuo to afford 9 as a white solid. Yield (1.86 g, 97% ); IR (CH₂Cl₂): νₘₐₓ=3373, 2983, 2574, 2461, 1724, 1413, 1369, 1235; ¹H NMR (270 MHz, CDCl₃): δ=4.48 (s, 2H, CH₂), 1.48 (s; 9H, Boc); MS (FAB⁺): m/z=214 [M+Na]⁺, 192 [M+H]⁺, 135, 123, 109, 69.

(Boc)aminooxy compound (10): N-hydroxysuccinimide (0.36 g, 3.13 mmol, 1 equiv), 9 (0.6 g, 3.13 mmol, 1 equiv), and N,N′-dicyclohexylcarbodiimide (0.68 g, 3.13 mmol, 1 equiv) were dissolved in EtOAc (90 mL), and the heterogeneous mixture was allowed to stir at room temperature overnight The mixture was then filtered through a pad of Celite® to remove the dicyclohexylurea, which was formed as a white precipitate (rinsed with 60 mL of EtOAc), and added to a solution of 8 (1.97 g, 3.13 mmol, 1 equiv) in THF (10 mL). A pH of 8 was maintained for this heterogeneous reaction by addition of triethylamine (6 mL). The resulting mixture was allowed to stir at room temperature overnight. On completion the mixture was filtered and the solvent was removed under reduced pressure to give after purification by flash-chromatography (CH₂Cl₂MeOH/NH₃ 92:7:1) 10 as a white solid. Yield (2.3 g, 90% ); ¹H NMR (270 MHz, CDCl₃): δ=5.33-5.35 (m, 1H, H6′), 4.4-4.52 (m, 1H, H3′), 4.3 (s, 2H, H90, 3.2-3.42 (m, 8H, H1, H2, H4, H6), 2.23-2.35 (m, 2H, H4′), 1.7-2.1 (m, 7H, H2′, H7′, H8′, H5), 1.44-1.46 (m, 18H, 2 Boc), 1-1.73 (m, 21H, H1′, H9′, H11′, H12′, H14′-H17′, H22′-H25′), 0.98 (3H, s, H-19′), 0.85 (d, J=6.5 Hz, 3H, H21′), 0.83 (d, J=6.5 Hz, 6H, H26′&H27′) and 0.65 (s, 3H, H18′); MS (FAB⁺): m/z=803 [M+H]⁺, 703 [M−Boc]⁺, 647, 603 [M−2Boc]⁺, 369, 279, 255, 235, 204, 145, 95, 69.

Hydroxylamine (11): To a solution 10 (1.1 g, 1.36 mmol, 1 equiv) in OH₂CH₂ (10 mL) was added TFA (2 mL, 20.4 mmol, 15 equiv) at 0° C. The solution was allowed to stir at room temperature for 5 hours. On completion toluene was added to azeotrope TFA from the reaction mixture. The solvents were removed In vacuo to afford after purification by chromatography (CH₂Cl₂/MeOH/NH₃ 92:7:1 to 75:22:3) 11 as a white solid (709 mg, Yield: 86% ); IR (CHCl₃): νₘₐₓ=3306, 2948, 2850, 2246, 1698, 1647, 1541, 1467, 1253, 1133; ¹H NMR (270 MHz, CDCl₃): δ=5.26-5.4 (m, 1H, H6′), 4.4-4.52 (m, 1H, H3′), 4.12 (s, 2H, H9), 3.34-3.41 (m, 2H, H2), 3.15-3.3 (m, 2H, H4), 2.6-2.74 (m, 4H, H1 & H6), 2.14-2.39 (m, 2H, H4′), 1.62-2.1 (m, 7H, H2′, H7′, H8′, H5), 1.02-1.6 (m, 21H, H1′, H9′, H11′, H12′, H14′-H17′, H22′-H25′), 0.96 (3H, s, H-19′), 0.86 (d, J=6.5 Hz, 3H, H21′), 0.83 (d, J=6.5 Hz, 6H, H26′&H27′) and 0.66 (s, 3H, H18′); MS (FAB⁺): m/z=603 [M+H]⁺, 369[Chol]⁺, 160, 137, 109, 95, 81, 69, 55.

Mannosyl compound (12a): A solution of D-mannose (266 mg, 4.8 mmol) in Acetic aqueous Buffer (sodium acetate/acetic acid 0.1 M, pH 4, 7 mL) and a solution of 11 (290 mg, 0.48 mmol, 10 equiv) in DMF (7 mL) was mixed and stirred for 3 days at room temperature. The solvent was removed in vacuo by freeze drying and chromatography (CH₂Cl₂/MeOH/NH₃ 75:22:3) afforded the product 21 a white solid (233 mg, Yield: 65%). The purity was further confirmed by HPLC. The final product contained of the β-pyranose (82% ) form and α-pyranose (18% ) form that were not isolated but characterized in the mixture. MS (FAB⁺): m/z=765 [M+H]⁺, 787 [M+Na]⁺, 397, 369[Chol]⁺, 322, 240, 121, 109, 95, 81, 69,57. β-pyranose form. ¹H NMR (400 MHz, CD₃OD/CDCl3 [75/25]): δ=7.64-7.62 (d, ³J₁ₐ₋₂ₐ=7 Hz, 1H, H1a), 5.35-5.36 (m, 1H, H6′), 4.45-4.5 (s, 2H,. H9), 4.35-4.5 (m, 1 H, H3′), 4.19-4.24 (dd, 1H, H2ₐ, ³J₁ₐ₋₂ₐ=7.4 Hz, ³J₂ₐ₋₃ₐ=7.7 Hz), 3.81-3.9 (m, 1H, H3a), 3.73-3.8 (m, 2H, H4a, H6ₐₓa), 3.63-3.71 (m, 2H, H5a, H_{eq}6a), 3.34-3.42 (m, 2H, H2), 3.27-3.30 (m, 2H, H4), 3-3.08 (m, 2H, H1), 2.9-2.98 (m, 2H, H6), 2.25-2.35 (m, 2H, H4′), 1.78-2.07 (m, 7H, H2′, H7′, H8′, H5), 1.03-1.65 (m, 21H, H1′, H9′, H11′, H12′, H14′-H17′, H22′-H25′), 1.01 (3H, s, H-19′), 0.91 (d, J=6.5 Hz, 3H, H21′), 0.85 (d, J=6.5 Hz, 6H, H26′&H27′) and 0.69 (s, 3H, H18′); ¹³C NMR (400 MHz, CDCl₃/CD₃OD [25/75[): 12.33 (C18′), 19.20 (C21′), 19.74 (C19′), 21.91 (C11′), 22.91 (C27′), 23.17 (C26′), 24.67 (C23′), 25.07 (C15′), 27.37 (C5), 28.85 (C25′), 28.96 (C2′), 29.07 (C12′), 32.76 (C7′), 32.87 (C8′), 36.38 (C2), 36.78 (C20′), 37.09 (C1) 37.76 (C22′),37.95 (C1′), 38.4 (C4), 39.36 (C4′), 40.41 (C24′), 40.76 (C16′), 46.16 (C6), 51.19 (C9′), 57.19 (C17′), 57.75 (C14′), 64.62 (C6a), 70.19 (C2a), 70.58 (C4a), 72.12 (C3a), 72.37 (C5a), 73.11 (C9), 75.91 (C3), 123.39 (C6), 140.72 (C5′), 155.02 (C1a), 158.69 (NHCOOChol), 173.1 (C8); α-pyranose form: identical data except, ¹H NMR (400 MHz, CD₃OD/CDCl3 [75/25]): δ=6.90-6.88 (d, ³J₁ₐ₋₂ₐ=7 Hz, 1H, H1a), 5-5.05 (dd, 1H, H2a, ³J₁ₐ₋₂ₐ=7.3 Hz, ³J₂ₐ₋₃ₐ=7.6 Hz); ¹³C NMR (400 MHz, CDCl₃/CD₃OD [25/75]): 65.33 (C2a), 155.79 (C1a). ¹H NMR (400, CD₃OD/CDCl₃ [75/25]): (m, 1H, H3′) missing, underneath solvent peak; confirmed by ¹H NMR (300 MHz, DMSO): δ=4.67-4.82 (m, 1H, H3′). ¹³C NMR (400 MHz, CDCl/CD₃OD [25/75]): C1 missing, underneath MeOH peak confirmed by ¹H/¹³C correlation at 400 MHz, around 49. Proton resonance assignments were confirmed using ¹H gradient type DQF-COSY and TOCSY; ¹H/¹³C correlation and DEPT 135 were used to assign unambiguously the carbon resonances. α pyrannose form gave ¹J¹³C1a-H1a=177 Hz and β pyrannose form gave ¹J¹³C1a-H1a=167 Hz. ¹H phase-sensitive NOESY confirmed conformation.

Glucosyl compound (12b): This was prepared with a solution of D-glucose (150 mg, 0.82 mmol) and 11 (100 mg, 0.16 mmol) in a similar way to the preparation of 12a, stirred for 1 day and purified by chromatography (CH₂Cl₂/MeOH/NH₃ 75:22:3) to afford the product 12b as a white solid (103 mg, Yield: 82% ). The purity was further confirmed by HPLC. The final product contained of the α-pyranose (11% ) anomer and β-pyranos (89% ) anomer that were not isolated but characterized In the mixture. (FAB⁺): m/z=765 [M+H]⁺, 787 [M+Na]⁺, 391, 369 [Chol]⁺, 309, 290, 171, 152, 135, 123, 109, 95, 81, 69; β-pyranose form. (300 MHz, CDCl₃/CD₃OD [90/10]): δ=7.53-7.56 (d, J=5.6 Hz, 1H, H1a), 5.26-5.36 (m, 1H, H6′), 4.2-4.45 (m, 3H, H9, H3′), 4.05-4.15 (m, 1H, H2a), 3.34-3.85 (m, 5H, H6a, H3a, H5a, H4a), 2.9-3.4 (m, H2, H4, MeOH), 2.9-3.15 (m, 4H, H1, H6), 2.15-2.3 (m, 2H, H4′), 1.65-2 (m, 5H, H2′, H7′, H8′), 0.95-1.55 (m, 23H, H5, H1′, H9′, H11′, H12′, H14′-H17′, H22′-H25′), 0.93 (3H, s, H-19′), 0.84 (d, J=6.5 Hz, 3H, H21′), 0.78 (d, J=6.5 Hz, 6H, H26′&H27′) and 0.62 (s, 3H, H18′); α-pyranose form: identical data except, ¹H NMR (300 MHz, CDCl₃/CD₃OD [90/10]): δ=7.22-7.24 (d, J=6,61 Hz, 1H, H1a), 4.95-5.07 (m, 1H, H2a); ¹H NMR (300 MHz, CD₃OD): (m, 1H, H3′) missing, presumably underneath solvent peak; confirmed by ¹H NMR (300 MHz, DMSO): δ=4.7-4.86 (m, 1H, H3′)

Galactosyl compound (12c): This was prepared with a solution of D-galactose (50 mg, 0.27 mmol) and 11 (40 mg, 0.066 mmol in a similar way to the preparation of 12a, stirred for 1 day and purified by chromatography (CH₂Cl₂/MeOH/NH₃ 75:22:3) to afford the product 12c as a white solid (35 mg, Yield: 70% ). The purity was further confirmed by HPLC. The final product contained of the α-pyranose (15% ) form and β-pyranose (85%) form that were not isolated but characterized in the mixture. MS (FAB⁺): m/z 765 M+H]⁺, 588, 391, 369 [Chol]⁺, 322, 290, 165, 152, 135, 121, 109, 95, 81, 69; β-pyranose form. ¹H NMR (270 MHz, DMSO): δ=7.78-7.82 (m, 1H, NHCO of C8), 7.55-7.58 (d, J=7.2 Hz, 1H, H1a), 6.95-7.1 (m,, 1H, NHCOOChol), 5.25-5.37 (m, 1H, H6′), 4.2-4.43 (m, 3H, H9, H3′), 3.2-3.9 (m, H2a, H6a, H3a, H5a, H4a, OH), 2.9-3.18 (m, 4H, H2, H4), 2.4-2.65 (m, 4H, H1, H6), 2.15-2.3 (m, 2H, H4′), 1.67-2 (m, 5H, H2′, H7′, H8′), 0.92-1.6 (m, 23H, H5, H1′, H9′, H11′, H12′, H14′-H17′, H22′-H25′), 0.96 (3H, s, H-19′), 0.89 (d, J=6.5 Hz, 3H, H21′), 0.84 (d, J=6.5 Hz, 6H, H26′&H27′) and 0.65 (s, 3H, H18′). α-pyranose form: identical data except, ¹H NMR (270 MHz, DMSO): 6.86-6.88 (d, J=6 Hz, 1H, H1a) Glucuronic compound (12d): This was prepared with a solution of D-glucuronic acid, sodium salt monohydrate (30 mg, 0.128 mmol, 1.5 equiv) and 11 (50 mg, 0.08 mmol) in a similar way to the preparation of 12a, stirred for 1 day, purified by chromatography (CH₂Cl₂/MeOH/NH₃ 75:22:3) to afford the sodium salt of 12d as a white solid (41 mg, Yield: 60% ). The purity was further confirmed by HPLC. The final product contained of the α-pyranose (85% ) form and β-pyranose (15% ) form that were not isolated but characterized in the mixture. MS (FAB⁺): m/z 779 [M+H]⁺, 733, 588, 411, 369[Chol]⁺, 336, 290, 240, 214, 159, 145, 135, 121, 109, 95, 81, 69,55. β-pyranose form. ¹H NMR (300 MHz, CDCl₃/CD₃OD [75/25]): δ=7.51-7.53 (d, J=5.9 Hz, 1H, H1a), 5.25-5.33 (m, 1H, H6′), 4.2-4.45 (m, 3H, H9, H3′), 3.8-4.1 (m, 3H, H2a, H3a, H4a), 3.6-3.75 (m, 1H, H5a), 3.2-3.55 (m, H2, H4, MeOH), 2.7-3.15 (m, 4H, H1, H6), 2.18-2.32 (m, 2H, H4′), 1.62-2 (m, 5H, H2′, H7′, H8′), 0.9-1.6 (m, 23H, H5, H1′, H9′, H11′, H12′, H14′-H17′, H22′-H25′), 0.93 (3H, s, H-19′), 0.83 (d, J=6.5 Hz, 3H, H21′), 0.77 (d, J=6.5 Hz, 6H, H26′&H27′) and 0.6 (s, 3H, H18′)); α-pyranose form: identical data except, ¹H NMR (300 MHz, CD₃OD): δ=7.22-7.24 (d, J=6.3 Hz, 1H, H1a), 5-5.1 (m, 1H, H2a).

β-lactesyl compound (12): A solution of β-D-Lactose, containing 25-30% of a (1.13 g, 3.3 mmol) and 11 (200 mg, 0.33 mmol) in 14 mL of DMF/Acetic aqueous Buffer was stirred for 4 days at room temperature. The solvent was removed In vacuo by freeze-drying and chromatography (CH₂Cl₂/MeOH/NH₃ 75:22:3) afforded the product 12e as a white solid (145 mg, Yield: 47% ). The purity was further confirmed by HPLC. The final product contained of the α-pyranose (15% ) form and β-pyranose (85% ) form (containing, itself around 25% of a lactose) that were not isolated but characterized in the mixture. MS (FAB⁺): m/z=927 [M+H]⁺, 588, 482, 369[Chol]⁺, 290, 243, 216, 178, 152, 135, 121, 109, 95, 81, 69,55; β-pyranose form. ¹H NMR (400 MHz, CDCl₃/CD₃OD [20/80]): δ_{H}=7.69-7.71 (d, ³J₁ₐ₋₂ₐ=5.8 Hz, 1H, H1a of β lactose), 7.66-7.68 (d, ³J₁ₐ₋₂ₐ=6.2 Hz, 1H, H1a of α lactose), 5.35-5.37 (m, 1H, H6′), 4.37-4.6 (m, 4H, H9, H3′, H2a), 4.2-4.37 (m, 1H, H1b), 3.65-4.05. (m, 7 H, H3a, H4a, H5a, H4b, H5b, H6b), 3.25-3.6 (m, 8H, H2, H4, H6a, H2b, H3b,MeOH), 3-3.2 (m, 4H, H1, H6), 2.25-2.42 (m, 2H, H4′), 1.8-2.15 (m, 5H, H2′, H7′, H8′), 1-1.65 (m, 23H, H5, H1′, H9′, H11′, H12′, H14′-H17′, H22′-H25′), 1.01 (3H, s, H-19′), 0.91 (d, J=6.5 Hz, 3H, H21′), 0.85 (d, J=6.5 Hz, 6H, H26′&H27′), and 0.69 (s, 3H, H18′); 13C NMR (400 MHz, CDCl₃/CD₃OD [20/80]): ¹³C NMR (400 MHz, CDCl₃/CD₃OD [20/80): 12.32 (C18′),19.2 (C21′), 19.76 (C19′), 21.94 (C11′), 22.91 (C27′), 23.17 (C26′), 24.7 (C23′), 25.1 (C15′), 27.22 (C5), 28.89 (C25′), 29 C2′), 29.1 (C12′) 32.8 (C7′), 32.92.(C8′), 36.29 (C22′),36.81 (C10′), 37.12 (C1′), 37.99 (C6), 38.11 (C1), 39.48 (C2), 40.45 (C24′), 40.80 (C16′), 46.13 (C4′), 51.23 (C9′), 57.22 (C17′), 57.80 (C14′), 62.41 (C6a), 63.4 (C6a), 70.02 (C5b), 70.63 (C2a), 72.8 (C3a), 73 (C3′), 73.18 (C9), 74,75 (C2b), 76.8 (C3a), 81 (C4b), 92.39 (C1b), 105.2 (C3′), 123.42 (C6′), 140.72 (C5′), 154.8 (C1a), 156.2 (NHCOOChol), 173.17 (C8). α-pyranose form: identical data except, ¹H NMR (400 MHz, CD₃OD/CDCl3 [80/20]): δ_{H}=7.04-7.05 (d, ³J₁ₐ₋₂ₐ=5.6 Hz, 1H, H1a), 5.05-5.07 (m, 1H, H2a), 4.09-4.11 (m, 1H, H3a); ¹H NMR (270 MHz, CD₃OD): (m, 1H, H3′) miissing, presumably underneath solvent peak; confirmed by ¹H NMR (300 MHz, DMSO): δ=4.7-4.85 (m, 1H, H3′). Proton resonance assignments were confirmed using ¹H gradient type DQF-COSY and TOCSY; ¹H/¹³C correlation and DEPT 135 were used to assign unambiguously the carbon resonances. ¹H phase-sensitive NOESY confirmed conformation.

Maltosyl compound (12f): This was prepared with a solution of D Maltose monohydrate (30mg, 1.8 mmol, 5 equiv) and 11 (100 mg, 0.16 mmol) in a similar way to the preparation of 12e, stirred for 1 day and purified by chromatography (CH₂Cl₂/MeOH/NH₃ 75:22:3) to afford 12f as a white solid (100 mg, Yield: 65% ). The purity was further confirmed by HPLC. The final product contained of the α-pyranose (87% ) form and β-pyranose (13% ) form that were not isolated but characterized in the mixture. MS (FAB⁺): m/z=927 [M+H]⁺, 765, 588, 559, 484, 369[Chol]⁺, 322, 290, 213, 167, 161, 143, 135, 121, 109, 95, 81, 69,55. β-pyranose form. ¹H NMR (300 MHz, CDCl/CD₃OD [80/20]): δ=7.55-7.57 (d, ³J₁ₐ₋₂ₐ=5.3 Hz, 1H, H1a), 5.3 (s, 1H, H6′), 4.85-5.02 (m, 1H, H3′), 4.09-4.22 (m, 1H, H1b), 3.57-4 (m, 7 H, H3a, H4a, H5a, H4b, H5b, H6b), 3.2-3.6 (m, 8H, H2, H4, H6a, H2b, H3b,MeOH), 2.8-3.1 (m, 4H, H1, H6), 2.1-2.36 (m, 2H, H4′), 1.6-2.05 (m, 5H, H2′, H7′, H8′), 1-1.6 (m, 23H, H5, H1′, H9′, H11′, H12′, H14′-H17′, H22′-H25′), 0.93 (3H, s, H-19′),: 0.83 (d, J=6.5 Hz, 3H, H21′), 0.78 (d, J=6.5 Hz, 6H, H26′&H27′) and 0.6 (s, 3H, H18′); α-pyranose form: identical data except. ¹H NMR(300 MHz, CD₃OD/CDCl3 [80/20]): δ=6.92-6.94 (d, J=4.62 Hz, 1H, H1a), 5.02-5.15 (m, 1H, H2a), 4.04-4.08 (m, 1H, H3a)

Maltotriosyl compound (12g): This was prepared with a solution of maltotriose (246.4 mg, 0.46 mmol, 7 equiv) and 11 (40 mg, 0.066 mmol) in a similar way to the preparation of 12e, stirred for 5 days and purified by chromatography (CH₂Cl₂/MeOH/NH₃ 75:22:3) to afford 12f as a white solid (61 mg, Yield: 85% ). The purity was further confirmed by HPLC. The final product contained of the α-pyranose (15% ) form and β-pyranose (85%) form that were not isolated but characterized in the mixture. MS (FAB⁺): m/z=1111 [M+Na]⁺, 1089 [M+H]⁺, 588, 423, 391, 369 [Chol]⁺, 240, 171, 159, 145, 121, 105, 95, 81, 69; β-pyranose form.: ¹H NMR (300 MHz, CDCl₃/MeOH[20/80]): δ=7.56-7.58 (d, J=6 Hz, 1H, H1a), 5.2-5.27 (m, 1H, H6′), 4.9-4.95 (m, 1H, H3′), 4.2-4.45. (m, 4H, H9, H3′, H2a), 4.05-4.2 (m, 2H, H1, H1c), 2.95-4 (m, 21H, H2, H4, H6a, H3a, H5a, H4a, H2b-6b, H2c-6c, MeOH), 2.85-2.95 (m, 4H, H1, H6), 2.2-2.3 (m, 2H, H4′), 1.8-2.1(m, 5H, H2′, H7′, H8′), 0.98-1.6 (m, 23H, H5, H1′, H9′, H11′, H12′, H14′-H17′, H22′-H25′), 0.94 (3H, s, H-19′), 0.84 (d, J=6.5 Hz, 3H, H21′), 0.78 (d, J=6.5 Hz, 6H, H26′&H27′) and 0.61 (s, 3H, H18′); α-pyranose form: identical data except, ¹H NMR (300 MHz, CDCl₃/MeOH[20/80]): δ=6.85 (d, J=5.6 Hz, 1H, H1a).

Maltotetraosyl compound (12h): This was prepared with a solution of D Maltotetraose (200 mg, 0.3030 mmol) and 11 (80 mg, 0.133 mmol, stirred for 5 days and purified by chromatography (CH₂Cl₂/MeOH/NH₃ 75:22:3) to afford 12 h as a white solid (67.5 mg, Yield: 41% ). The purity was further confirmed by HPLC. The final product contained of the α-pyranose (15% ) form and fpyranose (85% ) form that were not isolated but characterized in the mixture. MS (FAB⁺): m/z=1273 [M+Na]⁺, 1251 [M+H]⁺, 588, 369 [Chol]⁺, 159, 145, 121, 109, 95, 81, 69; HRMS (FAB⁺) C₅₉H₁₀₂N₄O₂₄Na: [M+Na]⁺ calcd 1273.6782, found 1273.6821. β-pyranose form.: ¹H NMR (300 MHz, CDCl₃/MeOH[20/80]): δ=7.56-7.58 (d, 1H, H1a), 5.15-5.25 (m, 1H, H6′), 4.95-5.1 (m, 1H, H3′), 4.38-4.5 (m, 4H, H9, H3′, H2a), 4.04-4.22 (m, 3H, H1b, H1c, H1d), 3.1-3.95 (m, 27H, H2, H4, H6a, H3a, H5a, H4a, H2b-6b, H2c-6c, H2d-6d, MeOH), 2.85-3.1 (m, 4H, H1, H6), 2.2-2.33 (m, 2H, H4′), 1.75-2.1 (m, 5H, H2′, H7′, H8′), 1-1.6 (m, 23H, H5, H1′, H9′, H11′, H12′, H14′-H17′, H22′-H25′), 0.92 (3H, s, H-19′), 0.82 (d, J=6.5 Hz, 3H, H21′), 0.78 (d, J=6.5 Hz, 6H, H26′&H27′) and 0.68 (s, 3H, H18′); α-pyranose form: identical data except, ¹H NMR (300 MHz, CDCl₃/MeOH[20/80]): δ=7 (d, 1H, H1a).

Maltoheptaosyl compound (12i): This was prepared with a solution of D Maltoheptaose (100 mg, 0.08673 mmol) and 11 (30 mg, 0.0497 mmol) stirred for 7 days and purified by chromatography (CH₂Cl₂/MeOH/NH₃ 75:22:3) to afford 121 as a white solid (46 mg, Yield: 53% ). The purity was further confirmed by. HPLC. The final product contained of the α-pyranose (15% ) form and β-pyranose (85% ) form that were not isolated but characterized in the mixture. MS (FAB⁺): m/z=1759 [M+Na]⁺, 1737 [M+H]⁺, 369 [Chol]⁺, 145, 121, 109, 95, 81. β-pyranose form.: ¹H NMR (300 MHz, CDCl₃/MeOH[20/80]): δ=7.53-7.58 (d, 1H, H1a), 5.35-5.37 (m, 1H, H6′), 4.97-5.12 (m, 1H, H3′), 4.45-4.6 (m, 4H, H9, H3′, H2a), 4-4.5 (m, 6H, H1b, H1c-g), 3.1-3.9 (m, 45H, H2, H4, H6a, H3a, H5a, H4a, H2b-6b, H2c-6c, H2d-6d, H2e-6e, H2f-6f, H2g-6g, MeOH), 2.7-3 (m, 4H, H1, H6), 2.15-2.35 (m, 2H, H4′), 1.7-2.1 (m, 5H, H2′, H7′, H8′), 1-1.6 (m, 23H, H5, H1′, H9′, H11′, H12′, H14′-H17′, H22′-H25′), 0.94 (3H, s, H-19′) 0.84 (d, J=6.5 Hz, 3H, H21′), 0.77 (d, J=6.5 Hz, 6H, H26′&H27′) and 0.63 (s, 3H, H18′); α-pyranose form: identical data except, ¹H NMR (300 MHz, CDCl₃/MeOH[20/80]): δ=6.9 (d, 1H, H1a).

### Biological and Biophysical Evaluation

General: Dioleoylphosphatidyl-ethanolamine (DOPE) was purchased from Avanti Lipid (Alabaster, Ala., USA). Plasmid pCMVβ was produced by Bayou Biolabs (Harahan, La., USA). DC-Chol was synthesised in our Laboratory^{[27]}. Mupeptde was synthesised by M. Keller by standard Fmoc based Merrifield solid phase peptide chemistry on Wang resine^{[43]}. All other chemicals were reagent grade.

Preparation of Liposomes: DC-Chol (7.5. mg, 15 μmol) and DOPE (7.5 mg, 10 μmol) were combined in dichloromethane. The solution was transferred to a round-bottomed flask (typically 50 ml) and organic solvent removed under reduced pressure (rotary evaporator) giving a thin-lipid film that was dried for 2-3 h in vacuo. Following this, 4 mM HEPES buffer, pH 7.2 (3 ml) was added to the round-bottomed flask so as to hydrate the thin-lipid film. After brief sonication (2-3 min.) under argon, the resulting cationic liposome suspension (lipid concentration of 5 mg/ml) was extruded by means of an extruder device (Northern lipid). Initially, three times through two stacked polycarbonate filters (0.2 μm) and then ten times through two stacked polycarbonate filters (0.1 μm) to form small unilamellar cationic liposomes (average diameter 105 nm according to PCS analysis). Lipid concentrations (approx. 44.8 mg/ml) were determined by Stewart assay^{[44]}.

Preparation of Liposome:Mu:DNA (LMD) and Liposome:DNA (LD) complexes: Initially, mu:DNA (MD) particles were prepared by mixing as follows. Plasmid DNA stock solutions (typically 1.2 mg/ml) were added to a vortex-mixed, dilute solution of mu peptide (1 mg/ml) In 4mM HEPES buffer, pH 7.2. The final mu:DNA ratio was 0.6:1 w/w, unless otherwise stated, and final plasmid DNA concentration was 0.27 mg/ml. MD containing solutions were then added slowly under vortex conditions to suspensions of extruded cationic liposomes (typically approx. 4.5 mg/ml), prepared as described above, resulting in the formation of small LMD particles with narrow size distribution (120±30 nm) as measured by PCS. Final lipid:mu:DNA ratio 12:0.6:1 w/w/w. A solution of sucrose (100%, w/v) in 4mM HEPES buffer, pH 7.2, was then added to obtain LMD particle suspensions in 4mM HEPES, buffer, pH 7.2 containing 10% w/v sucrose at the desired. DNA concentration (final DNA concentration typically 0.14 mg/ml) and the whole stored at −80° C. Liposome:DNA (LD) complexes (lipoplexes) were prepared for experiments with a lipid:DNA ratio of 12:1 (w/w) following the same protocol without the addition of Mu peptide.

Particle size measurements: The sizes of the lipoplexes were evaluated after 30 min exposure at 37° C. to biological media by Photon Correlation Spectroscopy (N4 plus, Coulter). The chosen DNA particular concentration was compatible with In vitro condition (1 μg/ml of DNA). The parameters used were: 20° C., 0.089 cP, reflexive Index of 1.33, angle of 90° C., 632.8 nm. Unimodal analysis was used to evaluate the mean particle size In Optimem. Size distribution program using the CONTIN algorithm was utilised to separate the sub-population of small serum particle of less than 50nm and to extracted the calculated size of lipoplexes in Optimem +10% FCS.

Transfection of HeLa cells: Cells were seeded In a 24-wells culture plate in DMEM supplemented with 10%. FCS and grown to approximately 70% confluence for 24 h at 37° C. in the presence of 5% CO₂. The cells were washed In PBS before the transfection media was administered to each well (0.5 ml of solution of 0, 50 or 100% Foetal Calf Serum In Dubelco OptiMem). 5 μl at 100 μg/ml DNA (nls βgal) of LMD were transfected. onto Hella Cells for 30 min. Cells were then rinsed 3 times with PBS and Incubated for a further 48 h in DMEM supplemented with 10% FCS prior to processing, for β-Gal expression by using standard chemiluminescent reporter gene assay kit (Roche Diagnostics, GmbH Cat No.1 758 241).

### Results and Discussion

Synthesis of Neoglycolipids: Each member of the targeted family of neoglycolipids consisted of a cholesterol bearing lipid and an oligosaccharide molecule bound together via a linker. The whole synthetic approach was divided in two parts; firstly the synthesis of a lipid containing the linker and secondly the chemioselective coupling of this lipid with chosen sugar molecules. The key to this strategy is the formation of a hydroxylamin (FIG. 1).

This synthesis of the Boo-protected lipid (8) was originally designed based on a convergent methodology using readily available aminoalcohols as starting materials with a complementary blocking group strategy for the amine group. This previously published methodology allowed the preparation of this polyamide-based lipid for gene transfer with little modification^{[27]}.

As mentioned, the glycosylation of hydroxylamino derivatives offers an elegant solution to our synthetic requirements. The commercially available O-(Carboxymethyl)hydroxyl-amine hydrochloride was first Boo-protected and then reacted with N-hydroxysuccinimide and N,N′-dicyclohexylcarbodiimide (DCC) resulting in the corresponding activated ester. This compound was treated immediately in situ with lipid (8) in THF at pH 8, affording a protected hydroxylamine. After a very straightforward deprotection with aqueous trifluoroacetic acid, the synthesis of the hydroxylamino lipid (11) was completed.

At this stage, we investigated the potential of our chemoselective coupling by reacting the lipid (11) with a number of commercially available non-protected oligosaccharides. This reaction was conducted under mild conditions using a solvent system of DMF and aqueous acetic acid pH 4 Buffer (1:1) which facilitates solubility of both sugar and lipid. As shown In FIG. 2 the reactants are in dynamic equilibrium with the open chair protonated intermediate. In order to force the equilibrium to product formation, an excess of sugar was added. Due to the amphiphilic nature of the neoglycolipid product, isolation during workup was found to be difficult as a result of micelle and foam formation. Solubility problems also hampered the isolation, purification and analytical process. Reaction times and yields varied depending on the carbohydrate used (Table 1).

TABLE 1
Yields, reaction times and diastereoselectivity of
glycosylation of product 11.

| Product | Sugar | Times (days) | Yield (%) | β/α |
|---|---|---|---|---|
| 12a | Mannose | 3 | 65 | 82/18 |
| 12b | Glucose | 1 | 80 | 89/11 |
| 12c | Galactose | 1 | 70 | 85/15 |
| 12d | Glucuronic acid | 1 | 60 | 85/15 |
| 12e | Lactose | 4 | 50 | 85/15 |
| 12f | Maltose | 1 | 65 | 87/13 |
| 12g | Maltotriose | 5 | 85 | 85/15 |
| 12h | Maltotetraose | 5 | 40 | 85/15 |
| 12i | Maltoheptaose | 7 | 55 | 85/15 |

Neoglycolid Conformation: Carbohydrate conformations can be ascertained by NMR in solution^{[28-33]}. The most useful data for conformation at the anomeric centre (C1a) is probably ¹J¹³C1a-H1a^{[34,35]}. The absolute value of this coupling constant depends upon the orientation of the carbon-hydrogen bond relative to the lone pairs of the ring oxygen, the electronegativity of the substituent at C1 and the nature of electronegative substituents attached to the rest of the molecule. The difference of ¹J¹³C1-H1 between a and β anomer of pyranoses can be used to determine the anomeric configuration. It is firmly established that ¹J(C1-H1eq)>¹J(C1-H1ax) with an approximate difference of 10 Hz. ¹J(C1-H1eq) is usually around 170 Hz and ¹J(C1-H1ax) approximately 160 Hz. Higher values are observed when O-1 is exchanged with more electronegative element as chlorine or fluorine but a 10 Hz difference is usually observed^{[36]}. Carbon-hydrogen coupling constants of furanosides have been investigated and ¹J(C1-H1eq)>¹J(C1-H1ax) but the difference is much smaller (1-3 Hz).

The characterization will be discussed based on the mannos example but the same analysis procedure was used for the other saccharides when NMR analysis conditions were favourable. Four distinct ring structures can be envisaged (FIG. 3). The pyranose forms can be reasonably expected to be favoured over the furanose rings for steric reasons. So out of the two observed compounds in NMR, the main one is probably a pyranose. The secondary observed compound could not be attributed to mutarotation equilibrium because phase sensitive NOESY did not show a cross peak between the two C1a signals (proving it Is a distinctive molecule). Therefore, this compound was not attributed to a furanose form because no shift of ¹³C5a was observed and ¹³C1a was not deshielded as has been demonstrated for related substituted furanose equivalents^{[33]}.

We measured ¹J¹³C1a-H1a=167 Hz for the main compound and ¹J¹³C1a-H1a=177 Hz for the secondary one. The absolute value of those ¹J¹³C1-H1 is 10 Hz higher than expected for classical ⁴C₁ conformation but this is explained by the extreme electronegativity of the O-substituted hydroxylamine group that could slightly deform the chair structure. For pyranose rings it has been established that [¹J(C1-H1eq)-¹J(C1-H1ax)]≈10 Hz, therefore it can be easily concluded that the main compound is the β anomer (H1ax) and the secondary compound is the α anomer (H1eq).

¹H phase sensitive NOESY confirmed this conclusion. Nuclear Overhauser effect was observed between H1a and H2a & H3a for the main compound. Considering the above detailed structure, this compound could not be the α pyranosyl anomer because the equatorial H1 cannot interact. In space with H3, whereas the β anomer is perfectly able to generate such interactions. No nuclear overhauser effect was observed for H1a of the secondary compound but this could be due to a lack of sensitivity. Hence, in accordance with data from ¹J¹³C1-H1 and NOESY analyses,, we concluded that two mannos pyranose α/β forms (208/0) were produced.

The very similar anomeric (β/α) isomers ratio obtained for the neoglycolipids is not surprising (Table 1), all the sugars having an equatorial hydroxyl in C2 but mannose. The ratio obtained for this last compound is surprising because the β anomer is usually reported as sterically less favourable than the α one. A possible explanation is that this reaction could be driven by some secondary interactions (Hydrogen bonding) between the sugar and the hydroxylamine linker, stabilizing the β anomer (this is consistent with the observation that the NMR signal of the β anomer Is always much more deshielded than the α one). This anomeric mixture of synthesized glycolipids are not expected to affect greatly, the researched biological properties of the liposomal constructs, therefore we did not attempt the tedious separation of those diasteroisomers by preparative high pressure liquid chromatography.

Biological application: The glyco-modification of LMD was based on the natural ability of miscellar suspension to incorporate into lipid membranes^{[37,38]}. Firstly LMD were formulated following standard, protocol and secondly a suspension of synthesized neoglycolipids miscelles in Hepes Buffer 4mM pH 7 was added, to the LMD and incubated for 30 min at room temperature before usual −80° C. storage. Different percents of all the neoglycolipids produced were tested for stabilization effect but only the longer chain (maltotetraose 12 h and maltoheptaose 12i) exhibited significant properties at less than 10% (data not shown).

The stabilisation effect of neoglycolipid modified LMD was demonstrated by incorporation, of 7.5 molar % of compound 12h or 12i. Lipid layers of lipdsomes based formulation are known to aggregate after salt or serum exposure^{[11,39,40]}. This phenomenon can be followed by measuring the average particle size increase after-a fixed time; any stabilization of the LMD particle should be reflected in a reduction of this parameter. It was chosen to measure the size of the lipoplexes by Photon Correlation Spectroscopy (PCS) after 30 min exposure at 37° C. to OptiMem or OptiMem +10% FCS to mimic standard in vitro conditions. It was not, possible to analyse the effect with PCS at higher serum percentages, the conditions being too extreme to allow for the taking of meaningful measurements. FIG. 4 describes the percentage of size increase of those lipoplexes.

The results indicate a clear stabilisation of the particle between LMD and standard liposome formulation. Neoglycolipids introduction at 7.5% proved significantly beneficial in OptiMem and 10% serum. 12i ncorporation proved to be the most efficient. This result indicates the need of long carbohydrate chains to create efficient molecular brushes on top of those cationic lipid layers^{[41, Sheiko, 2001 #119]}.

Even if some degree of stabilization is demonstrated, usually it results in a reduction of the affinity of the positively charged LMD for the negatively charged cell membrane, inducing a drop in the transfection ability of the construct. However in this case, the in-vitro transfection results indicated an enhancement of the transfection efficiency due to neoglycolipid modification in both 0% and 50% Serum condition (FIG. 5). This result was attributed to a short range protective effect due to these neoglycolipids hindering short range van der waals based Interactions between lipid bilayers of similar polarities but not affecting the longer range charge interactions between oppositely charged membranes. The aggregation induced by serum being based primarily on interaction of LMD with negatively charged proteins^{[42]}, the beneficial effect of our neoglycolipids was also lowered with an increasing percentage of serum (no significant benefit in 100% serum).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry or related fields are intended to be within the scope of the following claims

### REFERENCES

1a. W F Anderson, Science,1992, 256,808.

2a. F D Ledley, Current Opinion in Biotechnology, 1994, 5, 626;

K F Kozarsky, et al, ibid, 1993, 3, 499;

Gordon, et al,. ibid, 1994, 5, 611.

3a. C P Hodgon, BioTech, 1995, 13,222.

4a. P L Feigner, et al, Proc Natl Acad Sci USA, 1987, 84, 7413;

Feigner, et al, Nature, 1989, 337, 387;

H -J Burger, et al, Proc Natl Acad Sci USA, 1992, 89, 2145.

5a. Malone, et al, Proc Natl Acad Sci USA, 1989, 86, 6077.

6a. M -Y Chiang, et al, J Biol Chem, 1991, 226, 18162.

7a. R J Debs, et al, J. Biol Chem, 1990, 265, 10189;

C Walker, et al, Proc Natl Acad. Sci USA, 1992, 89, 7915.

8a. A D Bangham, Hospital Practice, 1992, 27, 51.

9a. J -P, Behr, et al, Proc Natl Acad Sci USA, 1989, 86, 6982;

R Leventis, et al, Biochim Biophys Ada, 1990, 1023, 124.

10a. X Gao, et al, Gene Therapy, 1995, 2, 710.

11a. R Stribling, et al, Proc Natl Acad Sd USA, 1992, 89, 11277.

12a E W F WAlton, et at, Nature Genetics, 1993, 5, 135.

13a. X Gao, et at, Biochim Biophys Res Commun, 1991, 179, 280.

14a. J E Morgan, et al, Arch Biochem Biophys, 1986, 246, 225.

15a. A E Pegg, Biochem, 1986, 234, 249.

16a. H Staudinger, et al, Helv Chim Acta, 1919, 2, 635;

S Knapp, et al, J Org Chem, 1992, 67, 6239

17a. K Omura, et a,, Tetrahedron, 1978, 34, 1651.

18a. J K Guy-Ceaffey, et al, J Biol Chem, 1995, 270, 31391.

21a. a) X Gao, L. Huang, Gene Ther. 1995, 2,710-722, and references therein;

b) A D. Miller, R. G. Cooper, C. J. Ethendge, L Stewart In Microspheres, Microcapsules and Uposomes (Ed. R. Arshady), John Wiley & Sons, 1997, in press, and references therein; c) P. L Feigner, Hum. Gene Ther. 1996, 7,1791-1793.

22a. a) H. Farhood, N. Serbina, L. Huang, Biochim. Biophys. Acta 1995, 1235, 289-295;

b) J. Zabner, A J. Fasbender, T. Moninger, K A Poelling r, M. J. Welsh, J. Biol. Chem. 1995, 270, 18997-19007.

23a. H. Mitsui, L. G. Johnson, S. H. Randell, R. C. Boucher, J. Biol. Chem. 1997, 272, 1117-1126.

24a. E. W. F. W. Alton, P. G. Middleton, N. J. Caplen, S. N. Smith, D. M. Steel, F. M. Munkonge, P. K. Jeffery, D. M. Geddes, S. L Hart, R. Williamson, K. I. Fasold, A. D. Miller, P. Dickinson, B. J. Steverison, G. McLachlan, J. R. Dorin, D. J. Porteous, Nature Genetics 1993, 5, 135-142.

25a. a) N. J. Caplen, E. W. F. W. Alton,. P. G. Middleton, J. R. Dorin, B. J. Stevenson, X. Gao, S. R. Durham, P. K. Jeffery, M. E. Hodson, C. Coutelle, L. Huang, D. J. Porteous, R. Williamson, D. M. Geddes, Nature Medicine 1995, 1, 39-46;

b) G. J. Nabel, E. G. Nabel, Z -y. Yang, B. A. Fox, G. E. Plautz, X. Gao, L. Huang, S. Shu, D. Gordon, A. E. Chang, Proc. Natl. Acad. Sci. USA 1993, 90, 11307-11311.

26a.. Uposomes: a practical approach (Ed.: R. R. C. New), IRL Press, Oxford, 1990.

27a. J. H. Feigner, R. Kumar, C. N. Sridhar, C. J. Wheeler, Y. J. Tsai, R. Border, P. Ramsey, M. Martin, P. L. Felgner, J. Biol. Chem. 1994, 269, 2550-2561.

28a. K. Omura, D. Swem, Tetrahedron 1978, 34, 1651-1660.

29a. S. Knapp, J. J. Hale, M. Bastos, A. Molina, K. Y. Chen, J. Org. Chem. 1992 57, 6239-6256

30a. (a) H. Staudinger, J. Meyer, Helv. Chim. Acta 1919, 2, 635-646;

(b) E. Fabiano, B. T. Golding, M. M. Sadeghl, Synthesis 1987, 190-192;

(c) B. T. Golding, M. C. aSullivan, L. L. Smith, Tetrahedron Lett. 1988, 29, 6651-6654;

(d) Y. G. Gololobov, L. F. Kasukhin, Tetrahedron 1992, 48, 1353-1406;

(e) A. W. Johnson, W. C. Kaska, K. A. O. Starzewski, D. A. Nixon, Ylides and imines of Phosphorus (Ed.: A. W. Johnson), J. Wiley and Sons, New York, 1993, chap. 13, pp. 403-483.

31a. (a) J. E. Baldwin, R. M. Adlington, A. S. Elend, M. L. Smith, Tetrahedron 1995, 51, 11581-11594;

(b) R. M. Moriarty, S. M. Tuladhar, L. Guo, S. Wehrli, Tetrahedron Left 1994, 35, 8103-8106.

32a. E. R. Lee, J. Marshall, C. S. Siegel., C. Jiang, N. S. Yew; M. R. Nichols, J. B. Nietupski, R. J. Ziegler, M. B. Lane, K. X. Wang, N. C. Wan, R. K. Scheule, D. J. Harris, E. Smith, S. H. Cheng, Hum. Gene Ther. 1996, 7, 1701-1717.

33a. C. J. Wheeler, P. L Feigner, Y. J. Tsai, J. Marshall, L. Sukhu, S. G. Doh, J. Hartikka, J. Nietupski, M. Manthorpe, M. Nichols, M. Piewe, X. Liang, J. Norman, A. Smith, S. H. Cheng, Proc. Natl. Acad. Sci. USA 1996, 93, 11454-11459.

34a. J. K. Guy-Caffey, V. Bodepudi, J. S. Bishop, K. Jayaraman, N. Chaudhary, J. Biol. Chem. 1995, 270, 31391-31396.

35a. a) J. -P. Vigneron, N. Oudrhiri, M. Fauquet, L. Vergely, J. -C. Bradley, M. Basseville, P. Lehn, J. -M. Lehn, Proc. Natg. Acad. Sci. USA 1996, 93, 9682-9686;

b) J. G. Lewis, K -Y. Un, A. Kothavale, W. M. Flanagan, M. D. Matteucci, B. De Prince, R. A Mook Jr., R. W. Hendren, R. W. Wagner, ibid 1996, 93, 3176-3181;

c) D. Moradpour, J. I. Schauer, V. R. Zurawski Jr., J. R. Wands, R. H. Boutin, Biochem. Biophys. Res. Commun. 1996, 221, 82-88.

36a. X. Gao, L. Huang, Biochem. Biophys. Res. Commun. 1991, 179, 280-285.

### References Cont'

[1] M. Ogris, S. Brunner, S. Schuller, R. Kircheis, E. Wagner, Gene Therapy 1999, 6, 595.

[2] A. L. Bailey, S. M. Sullivan, Biochimica Et Biophysica Acta-Biomembranes 2000, 1468, 239.

[3] J. L. Coll, P. Chollet, E. Brambilia, D. Desplanques, J. P. Behr, M. Favrot, Hum Gene Ther 1999, 10, 1659.

[4] P. Erbacher, T. Betwinger, P. Belguise-Valladier, S. Zou, J. L. Coll, J. P. Behr, J. S. Remy, J Gene Med 1999, 1, 210.

[5] S. M. Zou, P. Erbacher, J. S. Remy, J. P. Behr, J Gene Med 2000, 2, 128.

[6] A. Bragonzi, G. Dina, A Villa, G. Calori, A Biffi, C. Bordignon, B. M. Assael, M. Conese, Gene Therapy 2000, 7, 1753.

[7] S. Li, M. A. Rizzo, S. Bhattacharya, L. Huang, Gene Therapy 1998, 5, 930.

[8] M. I. Papisov, Advanced Drug Delivery Reviews 1998, 32, 19.

[9] D. V. Devine, A. J. Bradley, Advanced Drug Delivery Reviews 1998, 32, 19.

[10] C. Kitson, B. Angel, D. Judd, S. Rothery, N. J. Severs, A. Dewar, L. Huang, S. C. Wadsworth, S. H. Cheng, D. M. Geddes, E. Afton, Gene Therapy 1999, 6, 534.

[11] S. Li, W. C. Tseng, D. B. Stolz, S. P. Wu, S. C. Watkins, L. Huang, Gene Therapy 1999, 6, 585.

[12] N. Duzgunes, S. Nir, Advanced Drug Delivery Reviews 1999, 40, 3.

[13] D. Needham, D. H. Kim, Colloids and Surfaces B-Biointerfaces 2000, 18, 183.

[14] I. M. Hafez, P. R. Cullis, Adv Drug Deliv Rev 2001, 47, 139.

[15] D. Kirpotin, K. L. Hong, N. Mullah, D. Papahadjopoulos, S. Zalipsky, Febs Letters 1996, 388, 115.

[16] M. N. Jones, Advanced Drug Delivery Reviews 1994, 13, 215.

[17] S. Medda, S. Mukhedjee, N. Das, K. Naskar, S. B. Mahato, M. K. Basu, Biotechnology and Applied Biochemistry 1993, 17, 37.

[18] S. Kawakami, J. Wong, A. Sato, Y. Hattori, F. Yamashita, M. Hashida, Biochimica Et Biophysica Acta-General Subjects 2000, 1524, 258.

[19] S. Kawakami, A. Sato, M. Nishikawa, F. Yamashita, M. Hashida, Gene Therapy 2000, 7, 292.

[20] S. Kawakami, F. Yamashfta, M. Nishikawa, Y. Takakura, M. Hashida, Biochemical and Biophysical Research Communications 1998, 252, 78.

[21] C. F. Liu, C. Rao, J. P. Tam, Journal of the American Chemical Society 1996, 118, 307.

[22] L. E. Canne, A. R. Ferredamare, S. K. Burley, S. B. H. Kent, Journal of the American Chemical Society 1995, 117, 2998.

[23] K. Rose, Journal of the American Chemical Society1994, 116, 30.

[24] F. Peri, P. Dumy, M. Mutter, Tetrahedron 1998, 54, 12269.

[25] F. Peri, L. Cipqlla, B. La Ferla, P. Dumy, F. Nicotra, Glycoconjugate Journal 1999, 16, 399.

[26] S. E. Cervigni, P. Dumy, M. Mutter, Angewandte Chemie-International Edition in English 1996, 35, 1230.

[27] R. G. Cooper, C. J. Etheridge, L. Stewart J. Marshall, S. Rudginsky, S. H. Cheng, A. D. Miller, Chemistry—a European Journal 1998, 4, 137.

[28] H. Vanhalbeek, Current Opinion in Structural Biology 1994, 4, 697.

[29] W. C. Kett, M. Batley, J. W. Redmond, Carbohydrate Research 1997, 299, 129.

[30] C. A Bush, Glycobiology 1999, 9, 185.

[31] C. A Bush, M. Martin-Pastor, A. Imberty, Annual Review of Biophysics and Biomolecular Structure 1999, 28, 269.

[32] K. Bock, H. Thogerson, Annu. Rep. NMR Spectroscopy 1982, 13, 3.

[33] K. Bock, H. Thogerson, Journal of Carbohydrate Chem. 1992, 7, 813.

[34] K. Bock, C. Pedersen, J. Chem. Soc. Perkin Trans II 1974, 293.

[35] K Bock, I. Lundt, C. Pedersen, Tetrahedron Letters 1974, 1037.

[36] P. Hansen, Prog. Nucl. Magn. Reson. Spectroscop. 1980, 14, 249.

[37] T. Ishida, D. L. Iden, T. M. Allen, Febs Letters 1999, 460, 129.

[38] S. Kanda, K. Inoue, S. Nojima, H. Utsumi, H. Wiegandt, Journal of Biochemistry 1982, 91, 2095.

[39] P. R. Dash, M. L. Read, L. B. Barrett, M. Wodfert, L. W. Seymour, Gene Therapy 1999, 6, 643.

[40] M. Malmsten, Colloids and Surfaces a-Physicochemical and Engineeding Aspects 1999, 159, 77.

[41] J. Klein, E., Kumacheva, D. Mahalu, D. Perahia, L. J. Fetters, Nature 1994, 370, 634.

[42] J. P. Yang, L Huang, Gene Ther 1997, 4, 950.

[43] B. Merrifield, Science 1986, 232, 341.

[44] J. C. Stewart, Analytical Biochemistry 1980, 104, 10.

1. A compound capable of acting as a cationic lipid, the compound comprises a cholesterol group and a carbohydrate moiety.

2. A compound according to claim 1 wherein the compound is of the formula
Chol-L-Carb
wherein Chol is a cholesterol group, L is an optional linker group and Carb is a carbohydrate moiety.

3. A compound according to claim 1 or 2 wherein the cholesterol group Is cholesterol.

4. A compound according to claim 2 wherein the cholesterol group is linked to the optional linker group via a carbamoyl linkage.

5. A compound according to any one of claims 1 to 4 wherein the linker group is a polyamine group.

6. A compound according to claim 4 wherein the polyamine group is a naturally occuring polyamine.

7. A compound according to claim 4 or 5 wherein the polyamine group contains at least two amines of the polyamine group are spaced from each other by an ethylene (—CH₂CH₂—) group.

8. A compound according to claim 5 wherein the polyamine is any one of spermidine, spermine or caldopentamine.

9. A compound according to any one of claims 1 to 8 wherein the carbohydrate moiety is a mono-saccharide.

10. A compound according to any one of claims 1 to 8 wherein the carbohydrate moiety is a sugar moiety.

11. A compound according to any one of claims 1 to 8 wherein the carbohydrate moiety is selected from mannose, glucose (D-glucose), galactose, glucuronic acid, lactose, maltose, maltotriose, maltotetraose, maltoheptaose and mixtures thereof.

12. A compound according to any one of claims 1 to 8 wherein the carbohydrate moiety is D-glucose.

13. A compound according to any one of claims 1 to 12 wherein the compound comprises from 1 to 7 carbohydrate moieties.

14. A compound according to claim 13 wherein the compound comprises one carbohydrate moiety.

15. A compound according to claim 1 wherein compound is of the formula
Chol-L-Carb
wherein Chol is cholesterol
L is a polyamine group and
Carb is a glucose, preferably D-glucose.

16. A compound according to any one of claims 1 to 15 in admixture with or associated with a nucleotide sequence.

17. A process of preparing a compound according to any one of claims 1 to 15 comprising reacting a compound comprising a cholesterol group and a polyamine with a saccharide.

18. A compound according to any one of claims 1 to 15 or a compound when prepared by the process of claim 17 for use in therapy.

19. Use of a compound according to any one of claims 1 to 15 or a compound when prepared by the process of claim 17 in the manufacture of a medicament for the treatment of genetic disorder or condition or disease.

20. A cationic liposome formed from the compound according to any one of claims 1 to 15 or a compound when prepared by the process of claim 17.

21. A method of preparing a cationic liposome comprising forming the cationic liposome from the compound according to any one of claims 1 to 15 or a compound when prepared by the process of claim 17.

22. A cationic liposome according to claim 20 or a cationic liposome as prepared by the method of claim 21 for use in therapy.

23. Use of a cationic liposome according to claim 20 or a cationic liposome as prepared by the method of claim 21 in the manufacture of a medicament for the treatment of genetic disorder or condition or disease.

24. A combination of a nucleotide sequence and any one or more of a compound according to any one of claims 1 to 15, a compound when prepared by the process of claim 17, a cationic liposome according to claim 20, or a cationic liposome as prepared by the method of claim 21.

25. A combination according to claim 24 for use in therapy.

26. Use of a combination according to claim 24 in the manufacture of a medicament for the treatment of genetic disorder or condition or disease.

27. A pharmaceutical composition comprising a compound according to any one of claims 1 to 15 or a compound when prepared by the process of claim 17 admixed with a pharmaceutical and, optionally, admixed with a pharmaceutically acceptable diluent, carrier or excipient.

28. A pharmaceutical composition comprising a cationic liposome according to claim 20 or a cationic liposome as prepared by the method of claim 21 admixed with a pharmaceutical and, optionally, admixed with a pharmaceutically acceptable diluent, carrier or excipient.

29. A compound or a cationic liposome substantially as described herein and with reference to any on of the Figures.

30. A process substantially as described herein and with reference to any one of the Figures.
